(19) United States
(12) Patent Application Publication
(10) Pub. No.: US 2026/0341460 A1
(43) Pub. Date: Oct. 29, 2026

(54) APPARATUS AND METHOD FOR ADJUSTING THE RANGE OF BIO TISSUE DESTRUCTION

(71) Applicant: UNIVERSITY-INDUSTRY COOPERATION GROUP OF KYUNG HEE UNIVERSITY, Yongin-si (KR)

(72) Inventors: Ki Joo PAHK, Seoul (KR); Jun Hong PARK, Seoul (KR); Jeong Min HEO, Seoul (KR); Hyojun KIM, Goyang-si (KR); Byung Chul LEE, Seoul (KR)

(21) Appl. No.: 19/120,437

(22) PCT Filed: Jun. 1, 2023
(86) PCT No.: PCT/KR2023/007489

§ 371 Date: Apr. 11, 2025

(30) Foreign Application Priority Data
Oct. 24, 2022 (KR) 10-2022-0137611
Mar. 21, 2023 (KR) 10-2023-0036631

(51) Int. Cl. G16H 20/40; A61B 34/10; A61N 7/02

(52) U.S. Cl. CPC G16H 20/40; A61B 34/10; A61N 7/02

(57) ABSTRACT

A method for operating an apparatus for destroying biological tissue operated by at least one processor includes acquiring learning data including information on characteristics of biological tissue, a pressure of focused ultrasound, a variable acoustic pressure application time point, and a range of destruction of the biological tissue, and training a model such that, when the learning data is input, a pressure of focused ultrasound, a variable acoustic pressure application time point, and a number of pulses are output depending on the characteristics of the biological tissue and the range of destruction of the biological tissue. The method includes setting a pressure of focused ultrasound, a variable acoustic pressure application time point, and a number of pulses, which are used to destroy target biological tissue, when a medical image including the target biological tissue is input into the trained model.

### TECHNICAL FIELD

The present invention relates to an apparatus for destroying biological tissue and a method for adjusting a range of biological tissue destruction using the same, and more specifically, to an apparatus and method for adjusting a range of biological tissue destruction by controlling the intensity of a shock wave scattering effect.

### BACKGROUND ART

Focused ultrasound technology can deliver powerful acoustic energy ranging from tens to hundreds of times the atmospheric pressure to a desired area in a body in a completely non-invasive manner. The acoustic energy can selectively deliver thermal or mechanical energy to a desired part of the body with a spatial resolution of several millimeters.

High-intensity focused ultrasound (HIFU) based on ultrasound heat energy is a technology that focuses high-intensity ultrasound energy in one place to generate high heat (e.g., 50 °C or higher) at a focal point of ultrasound, causing thermal necrosis of tissue. Currently, HIFU is widely used clinically for treatment of uterine fibroids, adenomyosis, benign prostatic hyperplasia, prostate cancer, metastatic bone tumor, and tremors.

In addition, recently, the effect of physically destroying biological tissue using focused ultrasound has been reported in addition to the thermal ablation effect using HIFU. This technology, called ‘boiling histotripsy’, is a technology of using pressure tens of times more powerful than the acoustic pressure used in the existing HIFU to artificially generate acoustic cavitation at the focal point of ultrasound, thereby physically destroying focal biological tissue.

At the current preclinical animal testing stage, the effectiveness of safely removing solid tumors using boiling histotripsy technology has been demonstrated. The boiling histotripsy technology has a shorter treatment time compared to the existing HIFU thermal ablation and enables physical destruction of biological tissue and treatment process monitoring through real-time cavitation monitoring, attracting attention as a promising next-generation ultrasound surgical technology that can replace the existing surgical method applied in clinical practice.

Although the boiling histotripsy ultrasound technique is well-suited for physically removing large lesions, cavitation is likely to occur not only in the focal region of ultrasound but also in the surrounding tissue due to the shock wave scattering effect that occurs during the boiling histotripsy process. Therefore, the boiling histotripsy technology has a drawback in that it is very difficult to apply in surgeries requiring precise and fine removal.

To overcome such a drawback, a technology has recently been developed that can finely destroy biological tissue using variable acoustic pressure focused ultrasound without the shock wave scattering effect. The pressure-modulated shockwave histotripsy technology artificially generates primary vapor bubbles at the focal point of ultrasound, and then sequentially lowers the focal acoustic pressure below the threshold at which the shock wave scattering effect occurs. This allows fine destruction of surrounding biological tissue by controlling only dynamics of the primary vapor bubbles without causing the shock wave scattering effect.

The pressure-modulated shockwave histotripsy technology does not generate the shock wave scattering effect, unlike the existing boiling histotripsy technology, thereby allowing fine destruction of biological tissues. However, it has a drawback in that it is difficult for a user to arbitrarily adjust a range of biological tissue destruction.

### DISCLOSURE

### Technical Problem

Accordingly, the present invention attempts to provide an apparatus for destroying biological tissue and a method for adjusting a range of biological tissue destruction using the same capable of non-invasively destroying biological tissue by controlling the intensity of a shock wave scattering effect, and adjusting a range and intensity of biological tissue destruction.

### Technical Solution

In order to achieve the above object of the present invention, according to one aspect of the present invention, there is provided a method for operating an apparatus for destroying biological tissue operated by at least one processor, the method including:
acquiring learning data including information on characteristics of biological tissue, a pressure of focused ultrasound, a variable acoustic pressure application time point, and a range of destruction of the biological tissue; training a model such that, when the learning data is input, a pressure of focused ultrasound, a variable acoustic pressure application time point, and a number of pulses are output depending on the characteristics of the biological tissue and the range of destruction of the biological tissue; and, setting a pressure of focused ultrasound, a variable acoustic pressure application time point, and a number of pulses, which are used to destroy target biological tissue, when a medical image including the target biological tissue is input into the trained model.

The method may further include, after the setting, outputting a focused ultrasound at the set pressure of focused ultrasound to the target biological tissue, thereby destroying the target biological tissue, and controlling an acoustic pressure and intensity of the focused ultrasound to be lowered at the variable acoustic pressure application time point.

In the destroying, the focused ultrasound may have a nonlinear ultrasound waveform having a shock wave pressure of 40 MPa or higher or a pressure of a maximum positive pressure of 30 MPa or higher and a maximum negative pressure of-10 MPa or lower, the focused ultrasound after controlling the intensity of the focused ultrasound to be at or below a set value may have any one of linear and nonlinear ultrasound waveforms having a shock wave pressure of lower than 40 MPa or a pressure of a maximum positive pressure of 30 MPa or lower and a maximum negative pressure of −10 MPa or higher, and there are one or more pulses where a pulse duration is set to be 0.1 ms to 100 ms, an ultrasound center frequency is set to be 0.1 MHz to 10 MHz, a pulse repetition frequency is set to be 0.1 MHz to 100 MHz, and a duty cycle is set to be 0.1% to 10%.

The setting may involve setting a plurality of the variable acoustic pressure application time points to be after a time point at which vapor bubbles are generated in the target biological tissue due to an output of the focused ultrasound.

In the method, a pressure of the focused ultrasound, which is output to the target biological tissue at each of the plurality of set variable acoustic pressure application time points, may be controlled to be different.

The training the model may involve training the model such that, when the learning data is input, the range of destruction of biological tissue is displayed according to the characteristics of the biological tissue, the pressure of the focused ultrasound, and the variable acoustic pressure application time point.

The method may include, after the setting, when a medical image of the target biological tissue is input, displaying the pressure of the focused ultrasound and the variable acoustic pressure application time point, which are used to destroy the target biological tissue, as well as a range of destruction of the target biological tissue in the medical image.

The method may further include: after the controlling the intensity to be lowered, acquiring a destruction image in which the target biological tissue is destroyed according to the setting, comparing a difference range between an actual destruction range extracted from the destruction image and a determined destruction range corresponding to the information on the range of destruction of the biological tissue, and retraining the model using the actual destruction range, the pressure of the focused ultrasound, the variable acoustic pressure application time point, and the characteristics of the biological tissue when the difference range exceeds a preset threshold range.

In order to achieve the above object of the present invention, according to another aspect of the present invention, there is provided is an apparatus for destroying biological tissue, the apparatus including: an ultrasound output unit configured to output a focused ultrasound; and a processor configured to control a pressure of the focused ultrasound and a variable acoustic pressure application time point, in which the processor sets time points at which the pressure of the focused ultrasound is lowered to be different depending on characteristics of target biological tissue, the pressure of the focused ultrasound, and a range of destruction of biological tissue.

The processor may train a model such that, when learning data including information on characteristics of biological tissue, a pressure of focused ultrasound, a variable acoustic pressure application time point, and a range of destruction of the biological tissue is input, a pressure of focused ultrasound, a variable acoustic pressure application time point, and a number of pulses are output depending on the characteristics of the biological tissue and the range of destruction of the biological tissue.

The processor may set a plurality of the variable acoustic pressure application time points to be after a time point at which vapor bubbles are generated in the target biological tissue due to an output of the focused ultrasound.

The processor may control a pressure of the focused ultrasound, which is output to the target biological tissue at each of the plurality of set variable acoustic pressure application points, to be different.

The processor may train the model such that, when the learning data is input, the range of destruction of biological tissue is displayed according to the characteristics of the biological tissue, the pressure of the focused ultrasound, and the variable acoustic pressure application time point.

The processor may perform control such that, when a medical image of the target biological tissue is input, a pressure of the focused ultrasound and a variable acoustic pressure application time point, which are used to destroy the target biological tissue, are displayed as well as a range of destruction of the target biological tissue in the medical image.

The processor may extract an actual destruction range from a destruction image of the target biological tissue destroyed according to a setting, compare a difference range between the extracted actual destruction range and a determined destruction range corresponding to the information on the range of destruction of the biological tissue, and retrain the model using the actual destruction range, the pressure of the focused ultrasound, the variable acoustic pressure application time point, and the characteristics of the biological tissue when the difference range exceeds a preset threshold range.

### Advantageous Effects

According to the present invention, the variable acoustic pressure application time point after generation of vapor bubbles can be arbitrarily adjusted. Therefore, the lesion can be destroyed by adjusting the shock wave scattering effect intensity in a customized manner according to a size of the lesion to be destroyed.

In addition, it is possible to non-invasively destroy tumors ranging from relatively small microtumors to large tumors.

### DESCRIPTION OF THE DRAWINGS

FIG. 1 shows an example of a process of removing biological tissue using a focused ultrasound soft tissue removal technology of the related art.

FIG. 2 shows an example of principles of shock wave scattering effect and bubble cloud generation in the focused ultrasound soft tissue removal technology of the related art.

FIG. 3 is a configuration diagram of an apparatus for destroying biological tissue according to an embodiment of the present invention.

FIG. 4 is a flowchart of an operation of an apparatus for destroying biological tissue according to an embodiment of the present invention.

FIG. 5 is a flowchart of an operation of the apparatus for destroying biological tissue, according to another embodiment of the present invention.

FIG. 6 shows an example of a screen provided according to another embodiment of the present invention.

FIG. 7 is a flowchart of retraining a model according to an embodiment of the present invention.

FIG. 8 is a configuration diagram of a computing system according to an embodiment of the present invention.

### MODE FOR INVENTION

In the following detailed description, only certain embodiments of the present invention have been shown and described, simply by way of illustration. As those skilled in the art would realize, the described embodiments may be modified in various different ways, all without departing from the spirit or scope of the present invention. Accordingly, the drawings and description are to be regarded as illustrative in nature and not restrictive. Like reference numerals designate like elements throughout the specification.

Throughout the specification, unless explicitly described to the contrary, the word “comprise”, and variations such as “comprises” or “comprising”, will be understood to imply the inclusion of stated elements but not the exclusion of any other elements.

Hereinafter, an apparatus for destroying biological tissue and a method for adjusting a range of biological tissue destruction using the same according to an embodiment of the present invention will be described with reference to the drawings. Before describing an embodiment of the present invention, a focused ultrasound soft tissue removal technology of the related art will be described with

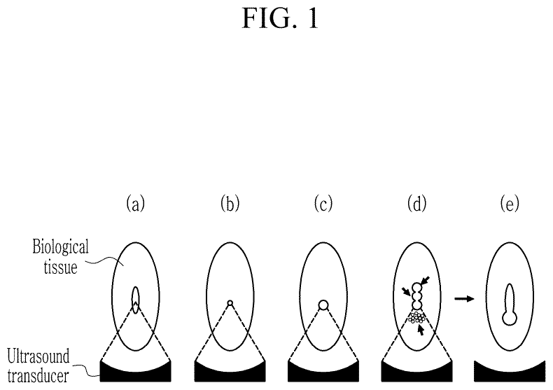

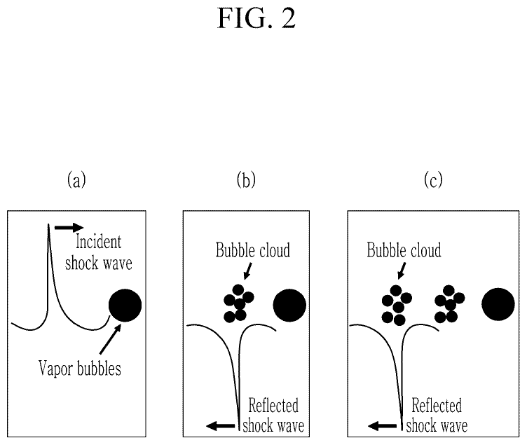

Reference to FIGS. 1 and 2.

FIG. 1 shows an example of a process of removing biological tissue using a focused ultrasound soft tissue removal technology of the related art.

Referring to FIG. 1, when high-intensity ultrasound is output using an ultrasound transducer to biological tissue to be removed (e.g., a lesion such as a tumor) as in (a) of FIG. 1, shock waves are generated at a focal point where the ultrasound is focused.

As shown in (b) of FIG. 1, when the moisture inside the tissue is heated by the thermal effect of the shock waves (shock wave heating), primary boiling vapor bubbles are generated at the focal point. As shown in (c) of FIG. 1, the vapor bubbles grow due to the shape of the shock wave waveform and the increase in vapor pressure inside the vapor bubbles caused by the vapor flowing into the bubbles.

In this way, cavitation occurs, in which microscopic cavities and bubbles are created within a medium due to ultrasound. In this case, these bubbles can generate shocks by oscillating and collapsing in response to changes in acoustic pressure. Boiling histotripsy is a technology of removing biological tissue through the shocks generated by such acoustic cavitation.

However, under general boiling histotripsy irradiation conditions, a shock wave scattering effect occurs due to the interaction between vapor bubbles and shock waves. As shown in (d) of FIG. 1, due to the shock wave scattering effect, numerous secondary bubble clouds are generated around the focal point.

The secondary bubble clouds can also shock at points other than the focal point, causing damage to areas of biological tissue that are not intended to be removed. Therefore, as shown in (e) of FIG. 1, a problem occurs in which the biological tissue is ultimately removed in a tadpole-shape rather than a circular shape.

FIG. 2 shows an example of principles of shock wave scattering effect and bubble cloud generation in the focused ultrasound soft tissue removal technology of the related art.

As shown in (a) of FIG. 2, the high-intensity focused ultrasound output by the ultrasound transducer generates a strong incident shock wave at the focal position. As shown in (b) of FIG. 2, the incident shock wave is reflected by the primary vapor bubble generated due to the thermal effect, and the reflected shock wave (hereinafter referred to as the ‘reflected shock wave’) overlaps with the incident shock wave to generate a stronger negative pressure field. When a negative pressure field is generated, it generates a bubble cloud composed of small vapor bubbles.

As shown in (c) of FIG. 2, the reflected shock wave continues to generate the scattering effect, resulting in the generation of numerous bubble clouds between the ultrasound transducer and the primary vapor bubbles.

The bubble clouds resulting from the unintended shock wave scattering effect can generate shocks at locations other than the focal point, causing damage to unintended areas of biological tissue. Therefore, the focused ultrasound soft tissue removal technology (boiling histotripsy) has only been used for removing biological tissue over a wide area and has been difficult to apply to surgeries requiring high precision, such as those around major blood vessels.

Thus, a technology has been studied which removes biological tissue by precisely controlling movement of vapor bubbles using variable intensity or variable acoustic pressure focused ultrasound without causing the shock wave scattering effect. However, this technology has a disadvantage in that it is difficult to control the range of biological tissue destruction because it removes biological tissue by controlling changes in the movement of vapor bubbles.

Therefore, an embodiment of the present invention provides a technique that can adjust the range and intensity of biological tissue destruction by controlling the intensity of shock wave scattering effect by adjusting the variable acoustic pressure application time point after primary vapor bubbles are generated.

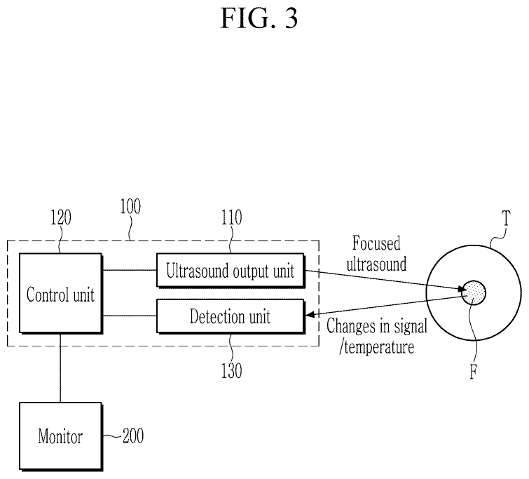

FIG. 3 is a configuration diagram of an apparatus for destroying biological tissue according to an embodiment of the present invention.

As shown in FIG. 3, an apparatus 100 for destroying biological tissue includes an ultrasound output unit 110, a control unit 120, and a detection unit 130. In addition, the apparatus 100 for destroying biological tissue may additionally include a monitor 200 or may be operated in conjunction with the monitor 200.

For reference, only the components necessary for describing the invention are shown in FIG. 3, and not all components essential for operating the apparatus 100 for destroying biological tissue or achieving the effects of the invention are shown. That is, in addition to the components shown in FIG. 3, additional hardware and/or software components may be further included to achieve the

### Effects of the Invention.

The ultrasound output unit 110 is composed of an ultrasound transducer capable of outputting focused ultrasound. An ultrasound transducer is a sound source that outputs ultrasound. An example is provided of one that outputs high-intensity ultrasound of 3 W/cm² (Ispta) or higher, but the present invention is not necessarily limited thereto.

The ultrasound output unit 110 in the embodiment of the present invention is described as being composed of a single transducer as an example. However, the ultrasound output unit may be configured as a transducer array in which a plurality of transducers focuses ultrasound onto a single focal point. That is, the ultrasound output unit is not limited to any particular form.

The ultrasound output unit 110 is connected to the control unit 130. When the ultrasound output unit 110 receives a control signal from the control unit 130 that determines characteristics of ultrasound, such as an acoustic pressure, a waveform, a frequency, and a variable acoustic pressure application time point, it outputs ultrasound with an intensity according to a set value based on the control signal.

In general, an ultrasound transducer converts alternating current energy of 20 kHz or higher into mechanical vibrations of the same frequency by applying the piezoelectric effect or magnetostrictive effect. For example, a transducer may have a structure including a body with one side open and piezoelectric elements, in which the inside of the body is filled with air, and a wire for applying a voltage is connected to each piezoelectric element.

For the piezoelectric element, a material that exhibits the piezoelectric effect, such as quartz and tourmaline, is used, and the transducer can generate and output ultrasound by utilizing the piezoelectric effect of the piezoelectric element. The structure of the transducer is only an example and is not limited to a specific structure or effect.

The piezoelectric element of the transducer can output ultrasound with an appropriate intensity by adjusting an output according to the area and purpose to be treated, and the output ultrasound overlaps to form an ultrasound beam. In addition, as shown in FIG. 3, the ultrasound output unit 110 irradiates high-intensity focused ultrasound to a biological tissue (T) where the lesion to be removed exists.

A focal point (F) is a point where the ultrasound beam output from the transducer is focused, and the vibration energy caused by the ultrasound is concentrated in one place to generate a powerful nonlinear shock wave. At the focal point (F), acoustic cavitation occurs in which vapor bubbles ranging in size from several hundred micrometers to several millimeters are generated due to the thermal effect of the shock wave. Additionally, biological tissue can be removed using the shock generated as the generated vapor bubbles oscillate and collapse/disappear (mechanical abrasion).

As described above, in boiling histotripsy, the shock wave scattering effect can occur due to the interaction between vapor bubbles and shock waves. The shock wave scattering effect generates numerous bubble clouds around the focal point, and the bubble clouds generate shocks at locations other than the focal point, causing damage to areas of biological tissue that are not intended to be removed.

That is, as shown in in FIG. 2 described above, the shock wave scattering effect occurs due to the interaction between high-intensity focused ultrasound and vapor bubbles. Therefore, an embodiment of the present invention proposes a technology that generates vapor bubbles at a focal point using focused ultrasound and then changes a variable acoustic pressure application time point to control the intensity of the shock wave scattering effect, thereby controlling a range of cavitation generation to adjust a range of destruction of biological tissue. In addition, an embodiment of the present invention proposes a technology that can destroy biological tissue in various ways according to characteristics of the biological tissue by setting the ultrasound intensity of focused ultrasound to be different depending on the variable acoustic pressure application time point.

That is, according to an embodiment of the present invention, it is possible to adjust a range of destruction of biological tissue depending on various variable acoustic pressure application time points. In other words, the apparatus 100 for destroying biological tissue may adjust the variable acoustic pressure application time point immediately after generating primary vapor bubbles.

Alternatively, the apparatus 100 for destroying biological tissue may apply the variable acoustic pressure application time point after a preset first time following generation of primary vapor bubbles. Alternatively, the apparatus 100 for destroying biological tissue may apply the variable acoustic pressure application time point after a preset second time following generation of primary vapor bubbles. Here, the second time may be longer than the first time.

By applying the variable acoustic pressure at various time points in this way, the apparatus 100 for destroying biological tissue can adjust the range of destruction of biological tissue. In the case of applying the variable acoustic pressure according to the example described above, the range of destruction is widest when the variable acoustic pressure is applied after the second time, second widest when applied after the first time, and narrowest when applied immediately after generating primary vapor bubbles.

In addition, in an embodiment of the present invention, during a process of performing a treatment to destroy biological tissue at a variable acoustic pressure application time point arbitrarily selected by an operator, the variable acoustic pressure application time point can be readjusted while monitoring the range of destruction of biological tissue being actually destroyed.

The detection unit 130 is a component that monitors whether a degree of destruction of biological tissue has been achieved as controlled by the control unit 120 when the biological tissue is destroyed by the focused ultrasound output from the ultrasound output unit 110. In an embodiment of the present invention, when the following two conditions occur, they are detected and transmitted to the control unit 120.

The first condition is a condition that vapor bubbles are generated in biological tissue while focused ultrasound is output to the biological tissue. The second condition is a condition that a temperature of the biological tissue reaches a preset threshold value.

In an embodiment of the present invention, the detection unit 130 may include a signal detection sensor for detecting a change in acoustic signal or electrical signal caused by the generation of vapor bubbles so as to detect whether the first condition is satisfied. Additionally, the detection unit 130 may include a temperature detection sensor for detecting a temperature change in biological tissue so as to detect whether the second condition is satisfied.

In an embodiment of the present invention, it is described that the detection unit 130 detects and transmits two conditions as an example to the control unit 120, but this is merely exemplary and at least one other condition may be set. In this case, various sensors suitable for detecting whether the condition is satisfied may be utilized.

When information on biological tissue for destruction is input, the control unit 120 sets a time point at which a pressure of the focused ultrasound is adjusted by controlling the ultrasound output unit 110. In this case, the control unit 120 can set the pressure and intensity of the focused ultrasound to be different at each time point depending on the time point at which the pressure of the focused ultrasound is adjusted.

Specifically, the control unit 120 determines the variable acoustic pressure application time point at which the pressure and intensity of the focused ultrasound are lowered to levels that do not generate a shock wave, based on information on the biological tissue for destruction that is input from the outside. The control unit 120 controls the intensity of the focused ultrasound to be at or below a set value by adjusting parameters such as an acoustic pressure of the ultrasound output from the ultrasound transducer through the ultrasound output unit 110, a focused ultrasound pressure, and an irradiation time.

Here, the set value refers to a maximum value of the intensity that prevents the focused ultrasound from causing the shock wave scattering effect within biological tissue. For example, when the preset time point is reached while the ultrasound output unit 100 is outputting high-intensity focused ultrasound having a nonlinear ultrasound waveform with a shock wave pressure of 40 MPa or higher at the focal point, or an acoustic pressure with a maximum positive pressure of 30 MPa or higher and a maximum negative pressure of −10 MPa or lower, the ultrasound output unit 100 adjusts the acoustic pressure of the focused ultrasound so that it becomes either a linear or nonlinear ultrasound waveform with a maximum positive pressure of lower than 40 MPa or 30 MPa or lower and a maximum negative pressure of −10 MPa or lower, thereby preventing the shock wave scattering effect and the generation of bubble clouds caused thereby.

The control unit sets a pulse duration to be 0.1 ms to 100 ms, an ultrasound center frequency to be 0.1 MHz to 10 MHz, a pulse repetition frequency to be 0.1 MHz to 100 MHz, and a duty cycle to be 0.1% to 10%. The number of pulses in this case is set to be 1 or more.

In this case, the control unit 120 uses focused ultrasound to generate vapor bubbles at the focal point, and then changes a variable acoustic pressure application time point at which the intensity of the shock wave scattering effect is minimized, thereby controlling the range of cavitation generation by minimizing or maximizing the intensity of the shock wave scattering effect.

To this end, the control unit 120 trains an artificial intelligence model (hereinafter, referred to as “variable acoustic pressure application time point determination model” for convenience of explanation) using learning data. The learning data is configured such that, when a range of destruction is input, information on the shock wave scattering effect intensity and the variable acoustic pressure application time point is output.

After training the variable acoustic pressure application time point determination model in this way, the control unit 120 receives a range of destruction input by an operator (e.g., a doctor) who operates the apparatus 100 for destroying biological tissue and outputs information on the shock wave scattering effect intensity and the variable acoustic pressure application time point. Alternatively, the control unit 120 that receives a patient's medical image can identify a size of biological tissue in the medical image and output information on the shock wave scattering effect intensity and the variable acoustic pressure application time point depending on the identified size of the biological tissue.

In addition, before the actual focused ultrasound is irradiated onto the biological tissue, the control unit 120 provides, through the monitor 200, the operator with an expected range of destruction in which the biological tissue is expected to be destroyed when the focused ultrasound is irradiated onto the biological tissue based on the output information on the shock wave scattering effect intensity and the variable acoustic pressure application time point. After checking the expected range of destruction provided through the monitor 200, the operator adjusts the range of destruction or operates the apparatus 100 for destroying biological tissue so that focused ultrasound is irradiated from the ultrasound output unit 110.

In addition, the control unit 120 may also adjust the pressure intensity of the focused ultrasound differently depending on the variable acoustic pressure application time point. That is, the control unit 120 can control the focused ultrasound to be output with a pressure intensity set to a first intensity at a first application time point and to a second intensity (higher or lower than the first intensity) different from the first intensity at a second application time point after the first application time point, depending on the variable acoustic pressure application time point.

This is because, depending on the characteristics of the biological tissue to be destroyed, some parts require repeated destruction, while other parts require only single destruction. Accordingly, the control unit 120 can control the ultrasound output unit 110 so that the focused ultrasound is output with different pressure intensities at each of the various variable acoustic pressure application time points.

Additionally, the control unit 120 acquires a biological tissue destruction image to be displayed on the monitor 200. Then, the control unit 120 compares a range in which the actual biological tissue is destroyed in the acquired biological tissue destruction image (hereinafter, referred to as the ‘actual destruction range’ for convenience of description) with a determined biological tissue destruction range (hereinafter referred to as the ‘determined destruction range’ for convenience of description) determined before the ultrasound is irradiated through the ultrasound output unit 110.

The control unit 120 determines the accuracy of the apparatus 100 for destroying biological tissue by comparing the actual destruction range with the determined destruction range. That is, the control unit 120 determines that the model is well-trained if a difference between the actual destruction range and the determined destruction range falls within a preset threshold range.

However, if the difference between the actual destruction range and the determined destruction range exceeds the threshold range, the model is retrained using the actual destruction range, the characteristics of the biological tissue, the variable acoustic pressure application time point, and the focused ultrasound pressure.

Here, since there are various methods for the control unit 120 to identify the actual destruction range from the biological tissue destruction image acquired via the monitor 200 and to compare the actual destruction range with the determined destruction range, an embodiment of the present invention is not limited to any particular method. In an embodiment of the present invention, an example is described in which the monitor 200 is photographed by a separate information collection means (e.g., a camera) (not shown) provided to the apparatus 100 for destroying biological tissue to acquire a biological tissue destruction image to be displayed on the monitor 200.

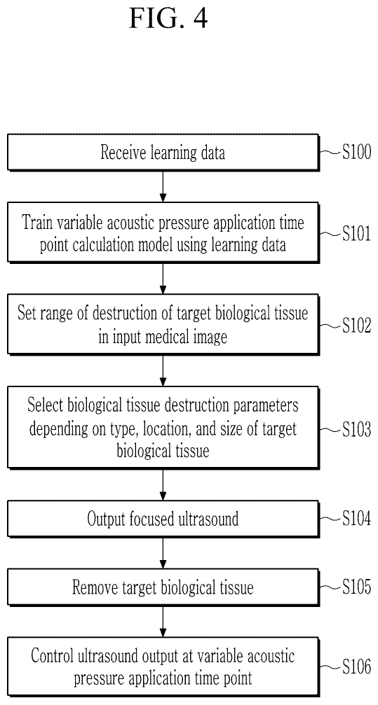

The operation of the apparatus 100 for destroying biological tissue will be described with reference to FIG. 4. In FIG. 4, an example is described in which the apparatus 100 for destroying biological tissue checks a medical image and automatically determines information on the shock wave scattering effect intensity and the variable acoustic pressure application time point.

FIG. 4 is a flowchart of an operation of the apparatus for destroying biological tissue according to an embodiment of the present invention.

As shown in FIG. 4, when collected learning data is input, the apparatus 100 for destroying biological tissue trains a model, which determines the variable acoustic pressure application time point, using the input learning data (S100, S101).

Here, the learning data includes a medical image with biological tissue, information on characteristics of the biological tissue, information on a range of destruction, and information on focused ultrasound pressure and variable acoustic pressure application time point, which are used to destroy the biological tissue. Accordingly, the apparatus 100 for destroying biological tissue trains the model to output the information on the focused ultrasound pressure and variable acoustic pressure application time point when the medical image, the information on characteristic of biological tissue, and the information on a range of destruction are input into the model, based on the learning data.

That is, the variable acoustic pressure application time point is set differently within a pulse duration after the first condition that vapor bubbles are generated occurs and is detected by the detection unit 130, depending on the characteristics of the biological tissue to be destroyed and the range of destruction of the biological tissue. Therefore, when such information is collected as learning information and used to train the model, the apparatus 100 for destroying biological tissue can provide the information on both the focused ultrasound pressure and the variable acoustic pressure application time point, as an output, when the medical image and the range of target biological tissue to be destroyed are input.

In other words, when a medical image of target biological tissue to be destroyed is input, the apparatus 100 for destroying biological tissue sets a range of destruction of the target biological tissue to be destroyed (S102). Since the apparatus 100 for destroying biological tissue can set a location of biological tissue in a medical image in various ways, the embodiment of the present invention is not limited to any particular method.

The apparatus 100 for destroying biological tissue that has set the range of destruction based on the medical image selects biological tissue destruction parameters, which includes the variable acoustic pressure application time point, a focused ultrasound pressure (hereinafter referred to as “first pressure”) before applying the variable acoustic pressure, a focused ultrasound pressure (hereinafter referred to as “second pressure”) immediately after applying the variable acoustic pressure, and a number of pulses, depending on a type of biological tissue as a target, a location and size of the biological tissue, and a range of destruction (S103). That is, the apparatus 100 for destroying biological tissue selects the variable acoustic pressure application time point or the variable acoustic pressure application time points for the first pressure and the second pressure based on one pulse, as well as the number of pulses in each section.

The apparatus 100 for destroying biological tissue controls the ultrasound output unit 110 based on the focused ultrasound pressure, the variable acoustic pressure application time points, and the number of pulses selected in step S103, and the ultrasound output unit 110 outputs focused ultrasound according to the first pressure (S104). The target biological tissue is removed according to the focused ultrasound output from the ultrasound output unit 110 (S105), and at the variable acoustic pressure application time point determined by the control unit 120, the ultrasound output unit 110 changes and outputs the focused ultrasound according to the second pressure (S106).

In this case, the apparatus 100 for destroying biological tissue can generate multiple time points regarding the variable acoustic pressure application time point. That is, from a time point the ultrasound is first output to a first application time point, the pressure intensity of the focused ultrasound is set high. At the first application time point, the pressure intensity of the focused ultrasound is lowered to reduce the shock wave scattering effect intensity. Then, at a second application time point, the pressure intensity of the focused ultrasound is increased again to enhance the shock wave scattering effect intensity. In this way, biological tissue can be destroyed using various pressure intensities of the focused ultrasound at different time points.

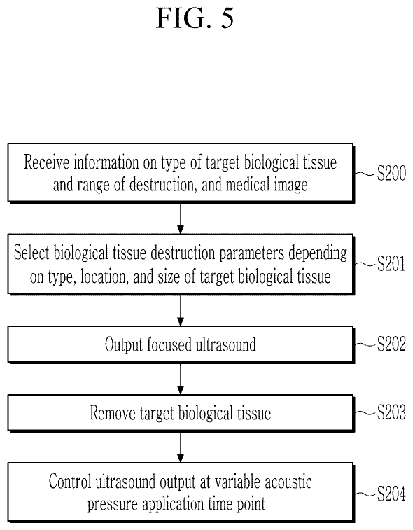

The above-described procedure describes a method by which the apparatus 100 for destroying biological tissue automatically sets the pressure intensity of input focused ultrasound and the variable acoustic pressure application time point in order to destroy biological tissue. However, when the operator sets the range of biological tissue, the apparatus 100 for destroying biological tissue can also set the variable acoustic pressure application time point and the pressure intensity of the focused ultrasound based on the set range. This will be described with reference to FIG. 5.

FIG. 5 is a flowchart of an operation of the apparatus for destroying biological tissue, according to another embodiment of the present invention.

As shown in FIG. 5, the apparatus 100 for destroying biological tissue receives information on a type of target biological tissue and a range of destruction and a medical image including biological tissue, which are input by an operator (S200). The apparatus 100 for destroying biological tissue selects biological tissue destruction parameters, which includes the variable acoustic pressure application time point, a focused ultrasound pressure (hereinafter referred to as “first pressure”) before applying the variable acoustic pressure, a focused ultrasound pressure (hereinafter referred to as “second pressure”) immediately after applying the variable acoustic pressure, and a number of pulses, depending on a type of biological tissue as a target, a location and size of the biological tissue, and a range of destruction, based on the received information (S201).

In this regard, an example is described in which the apparatus 100 for destroying biological tissue is trained in advance using learning data in order to select the first and second pressures, the variable acoustic pressure application time point, the number of pulses, and the like when the operator manually selects the range of destruction.

The apparatus 100 for destroying biological tissue controls the ultrasound output unit 110 based on the biological tissue destruction parameters selected in step S201, and the ultrasound output unit 110 outputs focused ultrasound according to the first pressure (S202). The target biological tissue is removed according to the focused ultrasound output from the ultrasound output unit 110 (S203), and at the variable acoustic pressure application time point determined by the control unit 120, the ultrasound output unit 110 changes and outputs the focused ultrasound according to the second pressure (S204).

In this case, the apparatus 100 for destroying biological tissue can generate multiple time points regarding the variable acoustic pressure application time point. That is, from a time point the ultrasound is first output to a first application time point, the pressure intensity of the focused ultrasound is set high. At the first application time point, the pressure intensity of the focused ultrasound is lowered to reduce the shock wave scattering effect intensity. Then, at a second application time point, the pressure intensity of the focused ultrasound is increased again to enhance the shock wave scattering effect intensity. In this way, biological tissue can be destroyed using various pressure intensities of the focused ultrasound at different time points.

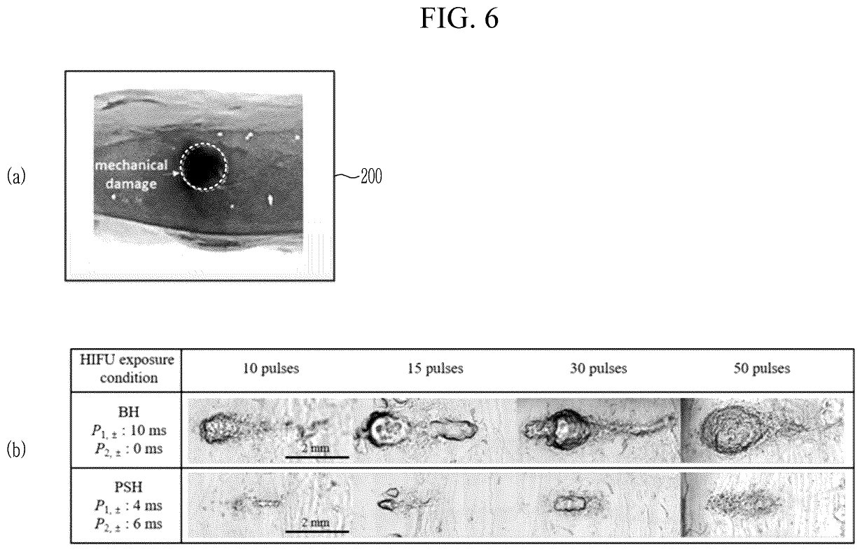

Here, when the apparatus 100 for destroying biological tissue selects the variable acoustic pressure application time point and the focused ultrasound pressure in step S202 of FIG. 5, the apparatus 100 for destroying biological tissue can display, through the monitor 200, a range of biological tissue to be destroyed based on the selected variable acoustic pressure application time point, focused ultrasound pressure, number of pulses, and the like. In an embodiment of the present invention, the variable acoustic pressure application time point, the focused ultrasound pressure, the number of pulses, and the like are referred to as the biological tissue destruction parameters, but are not necessarily limited thereto. This will be described with reference to FIG. 6.

FIG. 6 shows an example of a screen provided according to another embodiment of the present invention.

As shown in FIG. 6, the apparatus 100 for destroying biological tissue predicts and displays a range of destruction of biological tissue to be destroyed based on the selected biological tissue destruction parameters and displays the same, along with a medical image of target biological tissue to be destroyed, through the monitor 200 so that the operator can check them in real time.

In this case, the apparatus 100 for destroying biological tissue can display the range of destruction to the operator through the monitor, as shown in (a) of FIG. 6. Alternatively, as shown in (b) of FIG. 6, the apparatus 100 for destroying biological tissue may provide ranges of destruction of biological tissue to be destroyed based on various biological tissue destruction parameters according to each situation, and allow the operator to select the biological tissue destruction parameters based on the ranges.

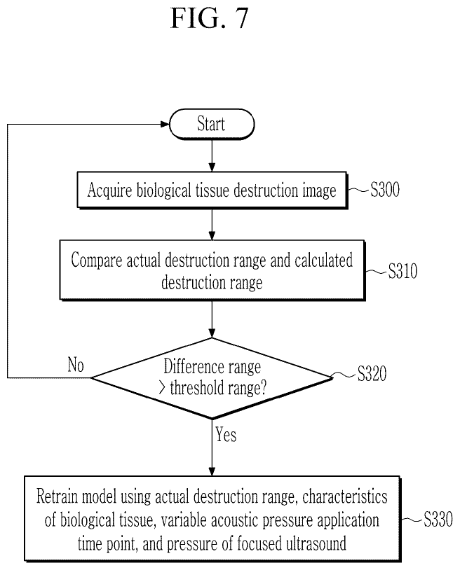

Here, the method by which the apparatus 100 for destroying biological tissue displays the range of destruction on the medical image can be applied using various technologies, and thus an embodiment of the present invention is not limited to any particular method. Next, a method of retraining a model by using the range of destruction displayed on the monitor 200 after the apparatus 100 for destroying biological tissue controls the ultrasound output at the variable acoustic pressure application time point in FIGS. 4 and 5 described above will be described with reference to FIG. 7.

FIG. 7 is a flowchart of retraining a model according to an embodiment of the present invention.

As shown in FIG. 7, the apparatus 100 for destroying biological tissue outputs focused ultrasound through the ultrasound output unit 110 and outputs focused ultrasound through controlled ultrasound output at the variable acoustic pressure application time point in steps S104 and S106 of FIG. 4 described above, thereby acquiring an image of destroyed biological tissue (S300). The monitor 200 on which a biological tissue destruction image is displayed can be collected through a photographing means (not shown; for example, a camera). Since there are various methods by which the apparatus 100 for destroying biological tissue acquires a biological tissue destruction image, an embodiment of the present invention is not limited to any particular method.

The apparatus 100 for destroying biological tissue compares the actual destruction range obtained from the acquired biological tissue destruction image with the determined destruction range determined before the ultrasound is irradiated through the ultrasound output unit 110 (S310).

The apparatus 100 for destroying biological tissue determines the accuracy of the apparatus 100 for destroying biological tissue by comparing the actual destruction range with the determined destruction range. That is, the apparatus 100 for destroying biological tissue determines whether the difference between the actual destruction range and the determined destruction range falls within a preset threshold range (S320).

It is determined that the model is well-trained if the difference between the actual destruction range and the determined destruction range falls within the threshold range. However, if the difference between the actual destruction range and the determined destruction range exceeds the threshold range, the model is retrained using the actual destruction range, the characteristics of the biological tissue, the variable acoustic pressure application time point, and the focused ultrasound pressure (S330).

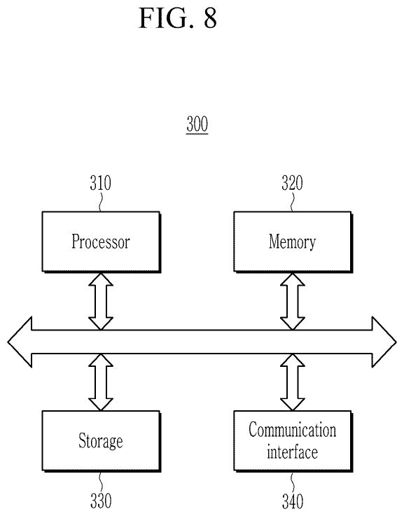

FIG. 8 is a configuration diagram of a computing system according to an embodiment of the present invention.

Referring to FIG. 8, the apparatus 100 for destroying biological tissue operated by at least one processor may be implemented as a computing system 300, and executes a program including instructions described to execute the operation of the present invention in the computing system 300. The program may be stored in a computer-readable storage medium and distributed.

The hardware of the computing system 300 may include at least one processor 310, a memory 320, a storage 330, and a communication interface 340, which may be connected via a bus. In addition, hardware such as input and output devices may be included. The computing system 300 may be equipped with various types of software, including an operating system capable of running a program.

The processor 310 is a device that controls the operation of the computing system 300 and may be various types of processors that process instructions included in the program, such as a central processing unit (CPU), a microprocessor unit (MPU), a micro controller unit (MCU), and a graphic processing unit (GPU).

The memory 320 loads the program so that the instructions described to execute the operation of the present invention are processed by the processor 310. The memory 320 may be, for example, a read only memory (ROM), a random access memory (RAM), and the like. The storage 330 stores various data, programs, and the like required to execute the operation of the present invention. The communication interface 340 may be a wired/wireless communication module.

Although the exemplary embodiments of the present invention have been described in detail, the scope of the present invention is not limited thereto, and various modifications and improvements made by one skilled in the art by using the basic concept of the present invention defined in the following claims also fall within in the scope of the present invention.

1. A method for operating an apparatus for destroying biological tissue operated by at least one processor, the method comprising:
acquiring learning data including information on characteristics of biological tissue, a pressure of focused ultrasound, a variable acoustic pressure application time point, and a range of destruction of the biological tissue;
training a model such that, when the learning data is input, a pressure of focused ultrasound, a variable acoustic pressure application time point, and a number of pulses are output depending on the characteristics of the biological tissue and the range of destruction of the biological tissue; and
setting a pressure of focused ultrasound, a variable acoustic pressure application time point, and a number of pulses, which are used to destroy target biological tissue, when a medical image comprising the target biological tissue is input into the trained model.

2. The method of claim 1, further comprising:
after the setting,
outputting a focused ultrasound at the set pressure of focused ultrasound to the target biological tissue, thereby destroying the target biological tissue, and
controlling an acoustic pressure and intensity of the focused ultrasound to be lowered at the variable acoustic pressure application time point.

3. The method of claim 2, wherein:
in the destroying,
the focused ultrasound has a nonlinear ultrasound waveform having a shock wave pressure of 40 MPa or higher or a pressure of a maximum positive pressure of 30 MPa or higher and a maximum negative pressure of −10 MPa or lower,
the focused ultrasound after controlling the intensity of the focused ultrasound to be at or below a set value has any one of linear and nonlinear ultrasound waveforms having a shock wave pressure of lower than 40 MPa or a pressure of a maximum positive pressure of 30 MPa or lower and a maximum negative pressure of −10 MPa or higher, and
there are one or more pulses where a pulse duration is set to be 0.1 ms to 100 ms, an ultrasound center frequency is set to be 0.1 MHz to 10 MHz, a pulse repetition frequency is set to be 0.1 MHz to 100 MHz, and a duty cycle is set to be 0.1% to 10%.

4. The method of claim 1, wherein:
the setting involves
setting a plurality of the variable acoustic pressure application time points to be after a time point at which vapor bubbles are generated in the target biological tissue due to an output of the focused ultrasound.

5. The method of claim 4, wherein:
a pressure of the focused ultrasound, which is output to the target biological tissue at each of the plurality of set variable acoustic pressure application time points, is controlled to be different.

6. The method of claim 1, wherein:
the training the model involves
training the model such that, when the learning data is input, the range of destruction of biological tissue is displayed according to the characteristics of the biological tissue, the pressure of the focused ultrasound, and the variable acoustic pressure application time point.

7. The method of claim 6, further comprising: further comprising,
when a medical image of the target biological tissue is input, displaying the pressure of the focused ultrasound and the variable acoustic pressure application time point, which are used to destroy the target biological tissue, as well as a range of destruction of the target biological tissue in the medical image.

8. The method of claim 2, further comprising:
after the controlling the intensity to be lowered,
acquiring a destruction image in which the target biological tissue is destroyed according to the setting,
comparing a difference range between an actual destruction range extracted from the destruction image and a determined destruction range corresponding to the information on the range of destruction of the biological tissue, and
retraining the model using the actual destruction range, the pressure of the focused ultrasound, the variable acoustic pressure application time point, and the characteristics of the biological tissue when the difference range exceeds a preset threshold range.

9. An apparatus for destroying biological tissue, the apparatus comprising:
an ultrasound output unit configured to output a focused ultrasound; and
a processor configured to control a pressure of the focused ultrasound and a variable acoustic pressure application time point, wherein
the processor sets the variable acoustic pressure application time point or the variable acoustic pressure application time points at which the pressure of the focused ultrasound is lowered to be different depending on characteristics of target biological tissue, the pressure of the focused ultrasound, and a range of destruction of biological tissue.

10. The apparatus of claim 9, wherein:
the processor trains a model such that, when learning data including information on characteristics of biological tissue, a pressure of focused ultrasound, a variable acoustic pressure application time point, and a range of destruction of the biological tissue is input, a pressure of focused ultrasound, a variable acoustic pressure application time point, and a number of pulses are output depending on the characteristics of the biological tissue and the range of destruction of the biological tissue.

11. The apparatus of claim 10, wherein:
the processor sets a plurality of the variable acoustic pressure application time points to be after a time point at which vapor bubbles are generated in the target biological tissue due to an output of the focused ultrasound.

12. The apparatus of claim 11, wherein:
the processor controls a pressure of the focused ultrasound, which is output to the target biological tissue at each of the plurality of set variable acoustic pressure application points, to be different.

13. The apparatus of claim 12, wherein:
the processor trains the model such that, when the learning data is input, the range of destruction of biological tissue is displayed according to the characteristics of the biological tissue, the pressure of the focused ultrasound, and the variable acoustic pressure application time point.

14. The apparatus of claim 13, wherein:
the processor performs control such that, when a medical image of the target biological tissue is input, a pressure of the focused ultrasound and a variable acoustic pressure application time point, which are used to destroy the target biological tissue, are displayed as well as a range of destruction of the target biological tissue in the medical image.

15. The apparatus of claim 14, wherein:
the processor extracts an actual destruction range from a destruction image of the target biological tissue destroyed according to a setting, compares a difference range between the extracted actual destruction range and a determined destruction range corresponding to the information on the range of destruction of the biological tissue, and retrains the model using the actual destruction range, the pressure of the focused ultrasound, the variable acoustic pressure application time point, and the characteristics of the biological tissue when the difference range exceeds a preset threshold range.
